(19) United States
(12) Patent Application Publication
(10) Pub. No.: US 2013/0338754 A1
(43) Pub. Date: Dec. 19, 2013

(54) RELEASE DEVICE FOR RELEASING A MEDICAL IMPLANT FROM A CATHETER AND CATHETER HAVING A RELEASE DEVICE AND IMPLANT FOR CONNECTION THERETO AND METHOD FOR RETAINING THE IMPLANT THEREIN

(71) Applicant: Biotronik AG, Buelach (CH)

(72) Inventor: Amir Fargahi, Buelach (CH)

(73) Assignee: Biotronik AG, Buelach (CH)

(21) Appl. No.: 13/894,015

(22) Filed: May 14, 2013

Related U.S. Application Data
(60) Provisional application No. 61/660,829, filed on Jun. 18, 2012.

(51) Int. Cl. A61F 2/95

(52) U.S. Cl. CPC A61F 2/95
USPC 623/1.11

(57) ABSTRACT

A release device for releasing a medical implant from an insertion device includes a retaining body for retaining the implant in the insertion device, having a proximal end, which is distant from a distal end of the insertion device, and a distal end, which faces the distal end of the insertion device, wherein the retaining body has at least one segment that radially delimits at least one space that extends between one of insertion elements and the at least one segment, wherein the implant has at least one retaining element at the proximal end thereof, for interaction with the retaining body, and wherein at least one region of the at least one retaining element, upon interaction with the retaining body, is inserted into the at least one space in a direction of insertion or is withdrawn from the at least one space against the direction of insertion.

### CROSS REFERENCE

The present application claims priority on copending provisional application No. 61/660,829 filed on Jun. 18, 2012; which application is incorporated herein by reference.

### TECHNICAL FIELD

Aspects of the present disclosure relate to a release device for releasing a medical implant from a catheter and a catheter comprising a release device for releasing a medical implant for implantation in an animal body and/or human body, and an implant for connection to is such a release device and a method for retaining the implant in such a release device.

### BACKGROUND

Implants are used often in medical applications for implantation in an animal body and/or human body permanently or at least for an extended period of time to perform replacement functions. Examples include cardiac pacemakers, brain pacemakers for Parkinson's patients, cardiac implants, cochlear implants, retinal implants, dental implants, joint replacement implants, vascular prostheses or stents.

Before introduction into the body, at least some implants are connected to catheters and must be fastened in such a way that they can be placed precisely at the application site without complication and can be released in a defined manner. To this end, it is known, for example, to equip the implant with eyes which interact with hooks on the catheter, thereby fastening the implant on the catheter.

### SUMMARY

One of the problems addressed by at least some embodiments of the invention is that of providing a release device with which an implant can be connected to an insertion device easily and in a user-friendly manner, and with which an implant can be released in a highly precise and targeted manner.

A further problem is that of providing a related insertion device.

Yet another problem is that of providing an implant for connection to such a related release device.

A further problem is that of providing a method for retaining the implant in such a release device.

These and other problems are solved according to embodiments of the invention by the features of the independent claims. Favorable embodiments and advantages of the invention will become apparent from the further claims and the description.

In one embodiment, a release device for releasing a medical implant from an insertion device is provided, in which the implant can be released at the distal end thereof first by way of a relative motion between a first insertion element and a second insertion element in a direction of insertion. The release device comprises a retaining body for retaining the implant in the insertion device, having a proximal end, which, in the state of use, is distant from a distal end of the insertion device, and a distal end, which, in the state of use, faces the distal end of the insertion device, wherein the retaining body has at least one segment that radially delimits at least one space that extends between one of the insertion elements and the at least one segment, wherein the implant has at least one retaining element at the proximal end thereof, at the least, for interaction with the retaining body, and wherein at least one region of the at least one retaining element, upon interaction with the retaining body, can be inserted into the at least one space in the direction of insertion and/or can be withdrawn from the at least one space against the direction of insertion.

Alternatively, it is provided that the implant can be released first at the proximal end thereof and comprises at least one retaining element at the distal end thereof, at the least, for interaction with the retaining body, and wherein at least one region of the at least one retaining element, upon interaction with the retaining body, can be inserted into the at least one space against the direction of insertion and/or can be withdrawn from the at least one space in the direction of insertion.

### DESCRIPTION OF THE DRAWINGS

The present disclosure is explained in the following in greater detail, as an example, with reference to exemplary embodiments that are depicted in drawings. They show, in a schematic representation:

FIG. 1A is a cross-sectional view through a favorable exemplary embodiment of an insertion device and a release device comprising an implant according to the present disclosure;

FIG. 1B is an enlarged side view of a retaining element of the implant shown in FIG. 1A;

FIG. 2 is an enlarged side view of a retaining body of the release device shown in FIG. 1A and a stopper of the insertion device;

FIG. 3 is an enlarged side view of the implant shown in FIG. 1A;

FIG. 4 is a cross-sectional view of the insertion device and the retaining body shown in FIG. 1A with the implant partially placed, before the outer insertion element is slid on;

FIG. 5 is a cross-sectional view of the insertion device and the retaining body shown in FIG. 1A with the implant completely placed, after the outer insertion element has been slid on;

FIG. 6 is a cross-sectional view of the retaining body shown in FIG. 5 with the implant connected thereto, in a detailed depiction;

FIG. 7 is a cross-sectional view of the insertion device and the retaining body shown in FIG. 1A with the implant completely released;

FIGS. 8 A-F illustrate a section through the retaining body shown in FIG. 1A along the line VII-VII (A) and five depictions through four alternative retaining bodies that have been cut in an analogous manner, as shown for A (B, E, F), and cut in a manner analogous to that depicted in FIG. 1A (C, D);

FIG. 9 is a cross-sectional view of an alternative insertion device and a retaining body with the implant partially placed, before an outer insertion element is slid on;

FIG. 10 is a cross-sectional view of the insertion device and the retaining body shown in FIG. 9, with the implant completely placed, after the outer insertion element has been slid on; and

FIG. 11 is an enlarged view of the insertion device and the retaining body shown in FIG. 9, with the implant partially released.

### DETAILED DESCRIPTION

By way of the embodiments according to the invention, it is possible to provide a release device in which the implant is securely held. In addition, a novel concept for retaining and releasing the implant is thereby made possible. The retaining body prevents unwanted movement of the implant, e.g. so-called jumping, when the implant is delivered to an implantation site and/or when the implant is released. The retaining element furthermore achieves a compact and efficient design of the release device. In addition, the particular release device permits easy handling and installation of the implant on the corresponding insertion device, such as a catheter, in the preparation laboratory. Moreover, release of the implant is reliable and rapid. Furthermore, it is possible to eliminate the risk of deformation of the implant and the resulting blockage of the particular release device, which occur with devices according to the prior art, which utilize hooks and eyelets. Such a retaining body is also particularly patient-friendly since it prevents the risk that hooks and eyes will be sheared by the implant, thereby releasing particles in the patient's body, as can happen with devices according to the prior art. Particularly advantageously, at least some of the embodiments according to the invention make it possible to reposition the partially released implant. This is the case in particular since the retaining body can generate a strong retaining force. Furthermore, the repositioning does not negatively affect the interaction between the retaining body and the at least one retaining element of the implant. In addition, the implant can be tested for functionality and, if a defect is found, the malfunctioning implant can be removed. The release of the implant according to one alternative embodiment by way of the proximal end first is particularly suitable for asymmetrical implants, which are used in cardiac catheter applications, for example. The invention also includes methods of using an apparatus of the invention, and it will be appreciated that in describing various apparatuses of the invention description of their use is also made.

In this context, a “retaining body” is intended to mean an element which holds another element, in particular, the implant, in a defined position on a segment of the insertion device, in particular, an insertion element or the internal shaft, by way of a clamping effect, a positive connection and/or a frictional connection. The retaining body can provide the retaining effect itself or it can cooperate with at least one further element. It can be, in particular, an element of the insertion device such as an insertion element or the internal shaft and/or an external shaft of the insertion device or a stopper or the implant. In addition, the retaining effect can be supported or induced by a material property of the retaining body or the other element. The term “induce” is intended to mean “generate, trigger and/or attain” in this case and in the remainder of the document.

A segment of the retaining body is intended to mean a region of the retaining body that is disposed with radial separation from an insertion element and, in particular, from the inner insertion element. As a result, a space is disposed radially between the internal insertion element and the at least one segment, or it extends between the inner insertion element and the at least one segment, wherein the at least one segment radially delimits the space on at least one side. The space is advantageously formed by a cavity and, in particular, by a cavity that is open on at least one side. Stable and unrestricted interaction between the at least one region of the retaining element and the retaining body can be achieved when the at least one space is located at the proximal end or at the distal end of the retaining body.

In this context, a retaining element of the implant refers to an element that provides a retaining effect in particular by way of a positive connection and/or a frictional connection. In addition, the retaining element is designed such that it can interact with the retaining is body. The retaining element comprises at least one change in direction that forms a total angle of more than 90°, preferably more than 120° and, particularly preferably, more than 150° relative to a region of the implant that extends substantially parallel to an inner axis of the implant, and is oriented in the radial direction with respect to the inner axis. The expression “substantially parallel” is intended to mean that a deviation of the direction of the region from the direction of the inner axis by up to 30° with respect to the parallel arrangement thereof is “parallel”. Furthermore, a total angle is defined as either an angle of a change in direction or as an angle formed by the sum of angles of two and/or more changes in direction. A change in direction refers, in particular, to a reversal of direction. The expression “oriented in the radial direction with respect to the inner axis” is intended to mean that the retaining element is disposed in a region radially between a wall of the implant and the inner axis. In particular, it does not mean that the region that can be inserted into or withdrawn from the space of the retaining body extends radially with respect to the inner axis.

In addition, the retaining element can be formed by many different suitable elements, such as a curved rod, a rod comprising a ball at the radially inner end, a prong or, preferably, a hook. In order to strengthen the retaining force, the retaining element can be designed, by way of a spring preload, for example, such that, when connected to the retaining body, it exerts a clamping force thereupon. Preferably, two and/or more retaining elements are distributed evenly over an outer surface or, in the case of a round implant, are distributed evenly over a periphery of the implant. Distribution that is not even would also be feasible in principle.

In this context, the direction of insertion is intended to mean the direction that extends from the proximal end of the insertion device to the distal end of the insertion device. In particular, the implant and the release device are introduced into the body using the insertion device in the direction of insertion. The terms “insertable” and/or “removable” are intended to mean a motion into the space and/or out of the space of the retaining body. The motion for insertion or removal can take place by way of direct action, by an assembler, for example, on the implant or by way of indirect action on the implant, via an insertion element, such as the outer insertion element. Furthermore, for insertion or is removal, the retaining body can also be moved relative to the implant, by way of the motion of an insertion element, such as the inner insertion element.

If the release of the implant starts at the distal end thereof, the release device can be used for a so-called distal insertion device. The inner insertion element (first insertion element) is connected to a tip of the insertion device, such as a catheter tip. The outer insertion element (second insertion element), which is disposed radially about the inner insertion element, is not connected to the tip, however, and can be moved relative to the tip by way of an axial motion in the direction of the proximal end of the insertion device. As a result, an implant disposed radially between the inner insertion element and the outer insertion element can be released via the distal end thereof first for the implantation or expansion thereof.

If the release device is designed according to one alternative embodiment in which the release of the implant starts at the proximal end thereof, the release device can be used for a so-called proximal insertion device. In this case, the outer insertion element is connected to the tip of the insertion device, but the inner insertion element is not. The outer insertion element is coupled to a guide element to permit movement thereof. The guide element extends coaxially to the inner guide element and inside thereof and is formed, for example, by a shaft having an insertion wire and a lumen. The guide element is connected to the proximal end of the insertion device to permit manipulation by an operator. The guide element is also connected to the tip. If the insertion element is now displaced in the direction of the distal end of the insertion device, it presses the tip and the outer insertion element in the distal direction, thereby permitting an implant that is radially disposed between the inner insertion element and the outer insertion element to be released by way of the proximal end first to permit the implantation or expansion thereof.

Advantageously, in a retained state, a positive connection between the segment of the retaining body and the at least one retaining element of the implant holds said implant in position, thereby preventing the implant from sliding out of the release device or the retaining body. In this case, a “retained state” means a state in which the implant is captively held in the insertion device.

Advantageously, the retaining body is fastened to one of the insertion elements and, in particular, to the inner insertion element. As a result, the retaining body and, therefore, the implant can be moved with the insertion element in a particularly captively-held manner during implantation. The space for the insertion or removal of the retaining element of the implant can be designed comfortably large when the retaining body is fastened by way of the proximal end thereof or the distal end thereof to one of the insertion elements and, in particular, the inner insertion element.

In an alternative or additional embodiment, the retaining body can be fastened to another element of the insertion device and, in particular, to the stopper that is disposed on the inner insertion element. This takes place, for example, by way of webs that are disposed and/or integrally formed at the proximal end of the retaining body.

Any type of connection deemed reasonable by a person skilled in the art, such as a frictional connection, a positive connection, or a bonded connection by way of welding, soldering, screws, chemical or heat bonding or attachment, pins, bolts, nails or adhesion, for example, can be used for both fastening variants. A fastening can be obtained using a efficient design when the retaining body is adhered to the inner insertion element and/or to the stopper. Advantageously, in some embodiments this is achieved using a UV-curable adhesive, thereby ensuring that an easily monitored and controlled procedure can be used.

In an advantageous embodiment, the retaining body comprises at least two segments, which radially delimit at least one space that extends between an inner insertion element and the segment in each case. The implant preferably comprises a corresponding number of retaining elements. The implant can therefore be connected to the retaining body in a particularly homogeneous and uniform manner. The segments and, therefore, the corresponding spaces are preferably distributed evenly over an outer surface or, in the case of a round retaining body, evenly over a periphery of the retaining body, or they are radially opposed with respect to the inner insertion element. Distribution that is not even would also be feasible in principle. The retaining body can also comprise more than two segments or associated spaces.

A particularly stable and reliable design of the retaining body can be achieved by forming the at least two segments on the same component. In other words, the at least two segments are designed to form one piece with each other. In this case, “one piece” is intended to mean that separating a first segment and a second segment from each other results in a loss of functionality of at least one of the segments. The component can have any cross-sectional shape, such as angular, triangular or quadrangular, dumb-bell-shaped, oval or, in particular, round.

Preferably, the at least two segments are disposed in a region of a component that extends in the circumferential direction about one of the insertion elements, in particular the inner insertion element. By way of this embodiment, the retaining element can be advantageously adapted to a round shape of the implant, thereby making it possible to establish an exact connection between the retaining body and the retaining elements. The component having the round cross-sectional shape can be formed by a tube, a cylinder and, particularly advantageously, by a bushing. A cylindrical jacket of the bushing preferably extends substantially parallel to the inner insertion element. The expression “substantially parallel” is intended to mean that a deviation of the direction of the segment from the direction of the inner insertion element by up to 30° with respect to the parallel arrangement thereof is “parallel”. Such a cylindrical jacket is commercially available, thereby making it possible to dispense with a complex design of a related component and save time and money. A base of the bushing, which extends radially about the insertion element, is preferably formed by an adhesive layer made of adhesive for fastening the retaining body on the insertion element.

In an alternative embodiment, the at least two segments are formed on two different components and/or are formed by two different components, thereby permitting each one to be matched individually to different geometries of the implant, such as length, curvature, etc., or to functions that deviate from one another, such as the simpler and, therefore, faster release of the retaining element or the like. The statements presented above with respect to segments designed as a single piece also apply for the segments described here.

In a preferred embodiment, the retaining body comprises a material selected from the group comprising a ceramic, a glass, a rubber, a plastic, a metal and a material that is visible to x-rays, thereby ensuring that a large number of materials that are easily accessible and are known in terms of the properties thereof can be used. Advantageously, the material is a plastic such as polyamide (PA) or acrylnitrile butadiene styrene (ABS). Such a material makes production of the retaining body easy since it is easily injected. They are therefore ideal for components having thin walls. Furthermore, these materials are biocompatible. Since these materials are unbreakable, a retaining body made of one of these materials can also be particularly resistant.

Alternatively, the material is a metal such as steel, Nitinol, tantalum, gold or platinum. As a result, a material that is resistant to bodily fluids in particular can be used. The metal is preferably a metal that is visible to x-rays, such as tantalum, gold or platinum, thereby providing the retaining body with a further function in a manner that saves space and assembly effort.

To increase the retaining force, a surface of the at least one segment of the retaining body that contacts the at least one retaining element of the implant, and/or a surface of the at least one retaining element of the implant that contacts the at least one segment of the retaining body can have a coating that serves to increase the friction between these components. To this end, the coating preferably comprises a material having high static friction in order to hold the implant in position in the retained state, thereby enabling the implant to be affixed using a efficient design. The material can be any of several suitable materials, such as a polymer and, in particular, a material selected from the group comprising polyamide, polyester, polyether block amide, silicone, polyurethane and, particularly preferably, a polyether block amide, such as PEBAX from the company Arkema. To ensure good contacting of the particular surface and the coating, a further layer or a connection layer can be provided, which is preferably made of a material that is suitable as a primer, such as the material linear low density polyethylene (LLDP).

According to a further embodiment of the invention, the retaining body comprises a passage for one of the insertion elements. This results in a compact configuration that is stabilizes and protects the insertion element extending therethrough. If the insertion device is a catheter, the applicable insertion element can be an inner shaft of the catheter.

According to a further aspect of the invention, an insertion device for inserting the medical implant is provided, which can be released at the distal end thereof first by way of a relative motion between a first insertion element and a second insertion element in a direction of insertion, comprising the release device for releasing the medical implant, comprising the retaining body for retaining the implant in the insertion device, having the proximal end, which, in the state of use, is distant from the distal end of the insertion device, and the distal end, which, in the state of use, faces the distal end of the insertion device, wherein the retaining body has at least one segment that radially delimits at least one space that extends between one of the insertion elements and the at least one segment, wherein the implant comprises the at least one retaining element at the proximal end therefore for interaction with the retaining body, and wherein the at least one region of the at least one retaining element, upon interaction with the retaining body, can be inserted into the at least one space in the direction of insertion and/or can be withdrawn from the at least one space against the direction of insertion.

In an alternative embodiment, the implant can be released first at the proximal end thereof and comprises at least one retaining element at the distal end thereof, at the least, for interaction with the retaining body, and wherein at least one region of the at least one retaining element, upon interaction with the retaining body, can be inserted into the at least one space against the direction of insertion and/or can be withdrawn from the at least one space in the direction of insertion.

By way of some embodiments according to the invention, it is possible to provide an insertion device in which the implant is securely held. Furthermore, unwanted movement of the implant, e.g. jumping, can be prevented when the implant is delivered to the implantation site and/or when the implant is released. The particular release device therefore has a compact and efficient design. In addition, the handling and assembly of the related insertion device, such as a catheter, in the preparation laboratory can be made efficient. Advantageously, the implant is released reliably and rapidly. Furthermore, the is blockage of the particular release device can be eliminated by way of the minimized risk of deformation of the implant. Particularly advantageously, some embodiments according to the invention makes it possible to reposition the partially released implant. In addition, the implant can be tested for functionality and, if a defect is found, the malfunctioning implant can be withdrawn. The insertion device according to the alternative embodiment, in which the proximal end of the implant is released first, can be used to particular advantage in implantations of asymmetrical implants such as cardiac catheter applications.

It is also provided that the insertion device comprises a stopper which limits a motion of the implant in the direction of a proximal end of the insertion device or in the direction of the distal end of the insertion device. Such a motion would disadvantageously result in blockage of the implant. According to a preferred development, the stopper induces ejection of the implant, and so the implant is released in an uncomplicated manner and without additional action by an operator of the release device, which unnecessarily extends the duration of implantation of an implant. Reference is made to the statements made above with respect to the definition of the term “induce”. The stopper can be made of any one of the materials mentioned above. The stopper preferably comprises a polymer as the material and, in particular, a polymer selected from the group comprising polyester, polyether block amide, silicone, polyurethane and polyamide. The stopper is preferably made of polyamide.

Embodiments of the invention furthermore relate to a medical implant comprising a base body for connection to the release device and for detachment from the insertion device, wherein the implant can be released at the distal end thereof first by way of a relative motion between the first insertion element and the second insertion element in the direction of insertion, comprising at least one retaining element for interaction with the retaining body, wherein the at least one retaining element has at least one change in direction, which u) forms a total angle of more than 90° relative to a region of the base body that extends substantially parallel to an inner axis of the base body, and is oriented in the radial direction with respect to the inner axis, and so the least one region of the at least one retaining element, upon interaction with the retaining body, can be inserted into the at least one space in the direction of insertion and/or can be withdrawn from the at least one space is against the direction of insertion.

Alternatively, the implant can be released at the proximal end thereof first and, upon interaction with the retaining body, at least one region of the at least one retaining element can be inserted into the at least one space of the retaining body against the direction of insertion and/or can be withdrawn from the at least one space in the direction of insertion.

By way of some embodiments according to the invention, an implant can be provided that can be securely held on the particular release device. In addition, a beneficial concept for retaining and releasing the implant is thereby made possible. By way of the at least one retaining element, unwanted movement of the implant, e.g. jumping, can be prevented when the implant is delivered to the implantation site and/or when the implant is released. Moreover, release of the implant is reliable and rapid. Furthermore, it is possible to eliminate the risk of deformation of the implant and the resulting blockage of the release device, which occur with devices according to the prior art, which utilize hooks and eyelets. Particularly advantageously, some embodiments according to the invention make it possible to reposition the partially released implant. In addition, the implant can be tested for functionality and, if a defect is found, the malfunctioning implant can be removed. The configuration and release of the implant according to an alternative embodiment by way of the proximal end first is particularly suitable for asymmetrical implants, which are used in cardiac catheter applications, for example.

The at least one retaining element is preferably disposed and/or integrally formed at (at least) the distal end or the proximal end of the implant. Furthermore, a “base body” in this context is intended to mean, a structure such as a wire mesh (or other) that substantially imparts a shape and/or a form to the implant, or forms the implant itself. By way of this structure, the base body has at least one basic structure. The at least one retaining element can be obtained in a manner having a particularly efficient design when it comprises the basic structure of the base body by at least more than 50%, preferably more than 75% and particularly preferably more than 90%. Advantageously, the at least one retaining element is formed by the basic structure of the base body.

According to a further advantageous embodiment, the at least one retaining element has a region that extends substantially in the radial direction toward the inner axis of the implant and is longer than a material thickness of the segment of the retaining body of the release device. As a result, interaction between the retaining body and the retaining element can preferably take place without friction. The interaction is therefore independent of an accuracy of dimensions of the retaining body and the at least one retaining element, and of tolerances in the production thereof, both of which are required in devices according to the prior art. The expression “substantially in the radial direction” is intended to mean that a deviation of the direction of the region from the radial direction toward the inner axis by up to 30° with respect to the perpendicular or radial arrangement thereof is “in the radial direction.”

According to an advantageous embodiment, the implant can be a self-expanding implant, whereby it can open automatically when released. Since the implant is self-expanding, an additional expanding means can be omitted. It is thereby possible, advantageously, to save space and assembly expense therefor. It is thereby also possible to simplify the design of the insertion device. It would also be possible, in principle, to use a balloon-expandable implant, however. The insertion device would have to be adapted accordingly for this purpose, however, which a person skilled in the art solves independently due to his technical knowledge.

Embodiments of the invention further relate to a method for the production of an implant comprising a base body, having at least one basic structure and at least one retaining element for interaction with a retaining body of a release device according to the invention, wherein the at least one retaining element comprises the basic structure by least more than 50%, preferably more than 75% and, particularly preferably, more than 90%, wherein the at least one retaining element is obtained by bending at least one segment of one end of the implant, in particular the distal end and/or the proximal end, radially in the direction of an inner axis of the implant or the base body. By way of one embodiment according to the invention, a stable and compact implant can be manufactured rapidly and in favorable manner with respect to expenditure.

Embodiments of the invention also relate to a method for connecting the medical implant by way of the retaining body of the release device in the insertion device, in which the implant is released at the distal end thereof first by way of a relative motion between the first insertion element and the second insertion element in the direction of insertion. The retaining body has the proximal end, which, in the state of use, is distant from the distal end of the insertion device, and the distal end, which, in the state of use, faces the distal end of the insertion device. The method comprises at least the following steps: place at least the proximal end of the implant, against the direction of insertion, at one of the insertion elements, in particular the inner insertion element, so that at least the proximal end of the implant is disposed proximally with respect to the retaining body; move at least the proximal end of the implant in the direction of insertion, so that at least the region of the retaining element of the proximal end of the implant is inserted, in the direction of insertion, into the space that is radially delimited by the retaining body; place the retaining body comprising the implant into at least one of the insertion elements, in particular the outer insertion element.

According to an alternative method, the implant is released at the proximal end thereof first and comprises at least these following steps: place at least a distal end of the implant, in the direction of insertion, at one of the insertion elements, so that at least the distal end of the implant is disposed distally with respect to the retaining body; move at least the distal end of the implant against the direction of insertion, so that at least one region of at least one retaining element of the distal end of the implant is inserted, against the direction of insertion, into a space that is radially delimited by the retaining body; place the retaining body comprising the implant into at least one of the insertion elements. The method according to the alternative embodiment, in which the proximal end of the implant is released first, is particularly suitable for the implantation of asymmetrical implants, which are used in cardiac catheter applications, for example.

By way of the embodiments according to the invention, a method can be achieved which enables a connection to be established between the implant and the release device in a manner that is user friendly, reliable and rapid. In the case of an implant that is connected to the release device in this manner, unwanted movement of the implant, e.g. jumping, can is be prevented when the implant is delivered to the implantation site and/or when the implant is released. In addition, the handling and assembly of the implant in the insertion device, e.g. a catheter, in the preparation laboratory is greatly simplified. Moreover, the implant can be released reliably and rapidly. Particularly advantageously, an implant that has been connected to the release device by way of some methods according to the invention and that is already partially released can be repositioned in a user friendly manner. In addition, a functionality test can be carried out on an implant that has been attached in this manner, and it can be removed if defective.

Preferably, at least the distal end of the implant is placed in the direction of insertion or the proximal end of the implant is placed against the direction of insertion at one of the insertion elements, in particular the inner insertion element, until the distal end or the proximal end impacts the stopper. The movement of at least the distal end against the direction of insertion or the proximal end of the implant in the direction of insertion takes place, for instance, by moving the outer tube against the direction of insertion or in the direction of insertion.

Furthermore, embodiments of the invention relate to a method for operating the release device comprising the retaining body, having the proximal end, which, in the state of use, is distant from the distal end of the insertion device, and the distal end, which, in the state of use, faces the distal end of the insertion device, and for releasing the medical implant from the insertion device in the direction of insertion, in which the implant is disposed between the first insertion element and the second insertion element, comprising at least the following steps: move at least the proximal end of the implant against the direction of insertion, so that at least one region of a retaining element of the proximal end of the implant is released against the direction of insertion from a space that is radially delimited by the retaining body; conclude the implantation.

According to an alternative method, it comprises at least the following steps: move at least the distal end of the implant in the direction of insertion, so that at least one region of a retaining element of the distal end of the implant is released in the direction of insertion is from a space that is radially delimited by the retaining body; conclude the implantation.

By way of some embodiments according to the invention, a method can be achieved which enables the implant to be released from the particular insertion device in a manner that is user friendly, reliable and rapid. In the case of an implant that has been released in this manner, unwanted motion of the implant, e.g. jumping, can be prevented when the implant is released. Furthermore, it is possible to eliminate the risk of deformation of the implant and a resulting blockage of the release device, thereby enabling the implant to be released reliably and rapidly. The release of the implant according to the alternative embodiment by way of the proximal end first is particularly suitable for asymmetrical implants, which are used in cardiac catheter applications, for example.

Preferably, the movement of at least the distal end of the implant and, therefore, the region of a retaining element in the direction of insertion is induced by way of a movement of one of the insertion elements, in particular the outer insertion element, in the direction of insertion, or the movement of at least the proximal end of the implant and, therefore, the region of a retaining element against the direction of insertion, is induced by a movement of one of the insertion elements, in particular the outer insertion element, against the direction of insertion. The movement of at least the distal end of the implant in the direction of insertion or of the proximal end of the implant against the direction of insertion continues until the distal end or the proximal end of the implant impacts the stopper. The implant can be expanded in two different ways. The implant can be moved, with the outer insertion element, in the direction of insertion or against the direction of insertion until the at least one retaining element is released from or emerges from the at least one space, whereupon the implant is expanded or automatically expands. Or, the at least one retaining element remains in the at least one space after the movement of the outer insertion element in the direction of insertion or against the direction of insertion, and is released by way of a to movement of the inner insertion element by a few millimeters, such as approximately 5 mm, against the direction of insertion or in the direction of insertion, whereupon the implant is expanded or automatically expands.

Various elements of invention embodiments as generally discussed above can be further illustrated through consideration of the figures. Turning now to those figures, elements that are functionally identical or similar-acting are labeled in the figures using the same reference characters. The figures are schematic depictions of the present disclosure. They do not depict specific parameters of the present disclosure. Furthermore, the figures merely show typical embodiments of the present disclosure and are not intended to limit the present disclosure to the embodiments shown. Before considering the figures, a list of reference element numbers used therein is provided for convenience:

### REFERENCE CHARACTERS

10 retaining element
12 End
14 End
16 Segment
18 Segment
20 Space
22 component
24 region
26 circumferential direction
28 component
30 bushing
32 cylindrical jacket
34 axial direction
36 passage
38 base
40 web
42 bar
50 shaft region
52 insertion element
54 insertion element
56 direction of insertion
100 release device
102 implant
104 retaining element
106 end
108 region
110 base body
112 directional change
114 region
116 inner axis
118 basic structure
120 region
122 end
125 catheter tip
130 insertion device
135 end
140 end
145 stopper
150 radial direction
155 opening
160 guide element
A distance
D outer diameter
L length
T material thickness
W wall thickness
A Angle
B Angle
Σ total angle

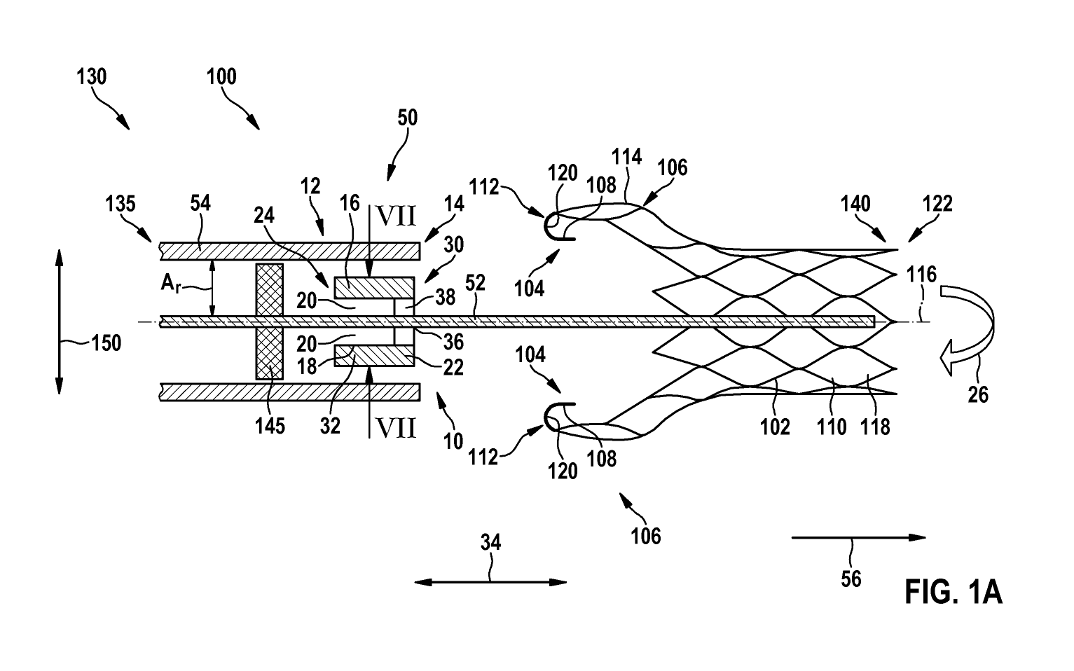

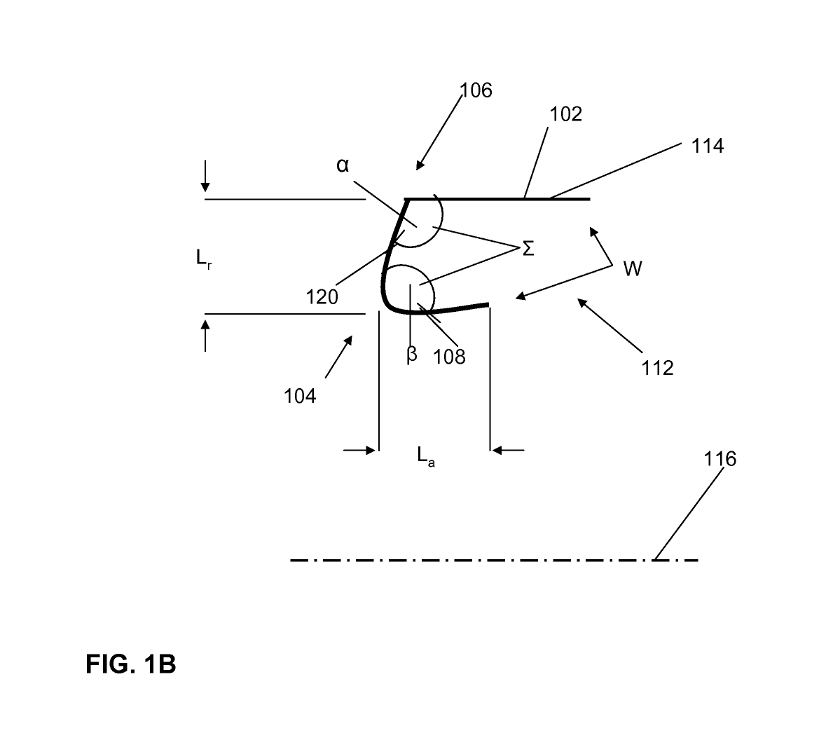

FIG. 1A shows a longitudinal sectional view through a favorable exemplary embodiment of a release device 100 of an insertion device 130, which is only partially depicted. The insertion device 130 is a catheter, for example, comprising a shaft region 50 having two coaxially disposed insertion elements 52, 54, e.g. an inner shaft (insertion element 52) and, enclosing same, an outer shaft (insertion element 54), which can be enclosed by an outer sleeve, which is not shown. In the state of use by an operator, i.e. during fastening of the implant 102 to the release device 100 or during implantation, the insertion device 130 faces an operator by way of the proximal end 135 thereof. The implant 102 is placed on the ic) distal end 140 of the shaft region 50 between the inner shaft and the outer shaft, and is intended for release at the implantation site in the animal body or human body.

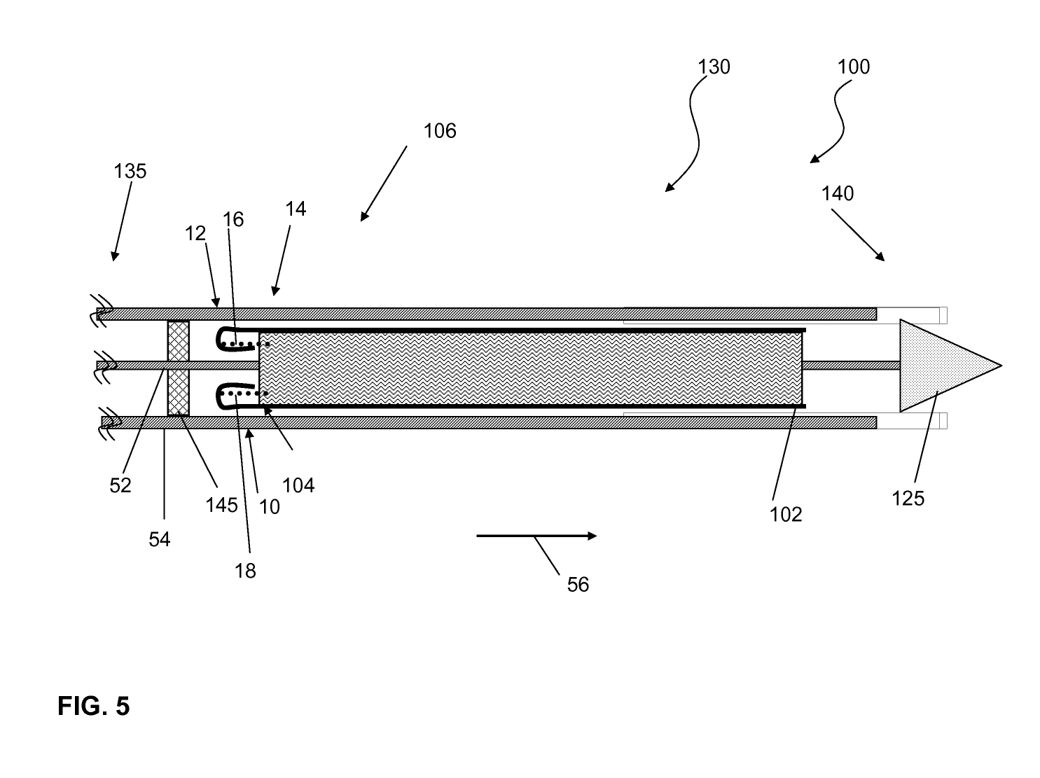

The release device 100 serves to release the medical implant 102 from the insertion device 130. The implant 102 is disposed at an end 140 of the shaft region 50 facing away from the is user, e.g. in the vicinity of a catheter tip 125 (see FIG. 5). The implant 102 is placed around the inner insertion element 52, for example, and is released in a direction of insertion 56 starting at a distal end 122 of the implant 102 by way of a relative motion between the first insertion element 52 and the second insertion element 54. The inner insertion element 52 is connected to the catheter tip 125, but the outer insertion element 54 is not.

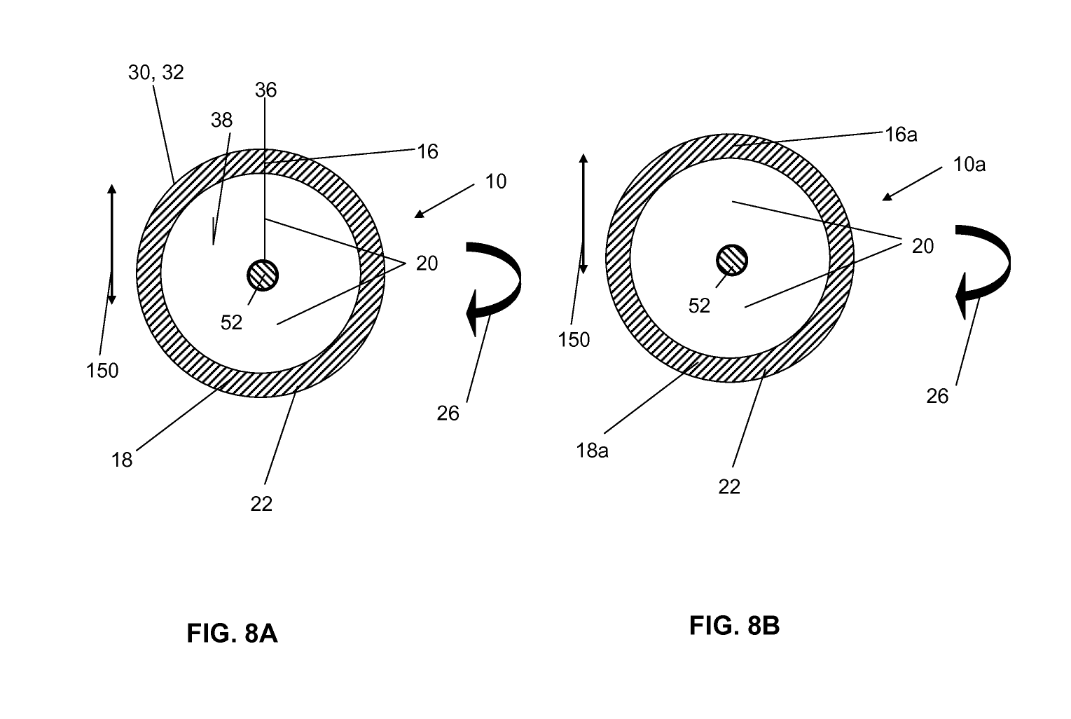

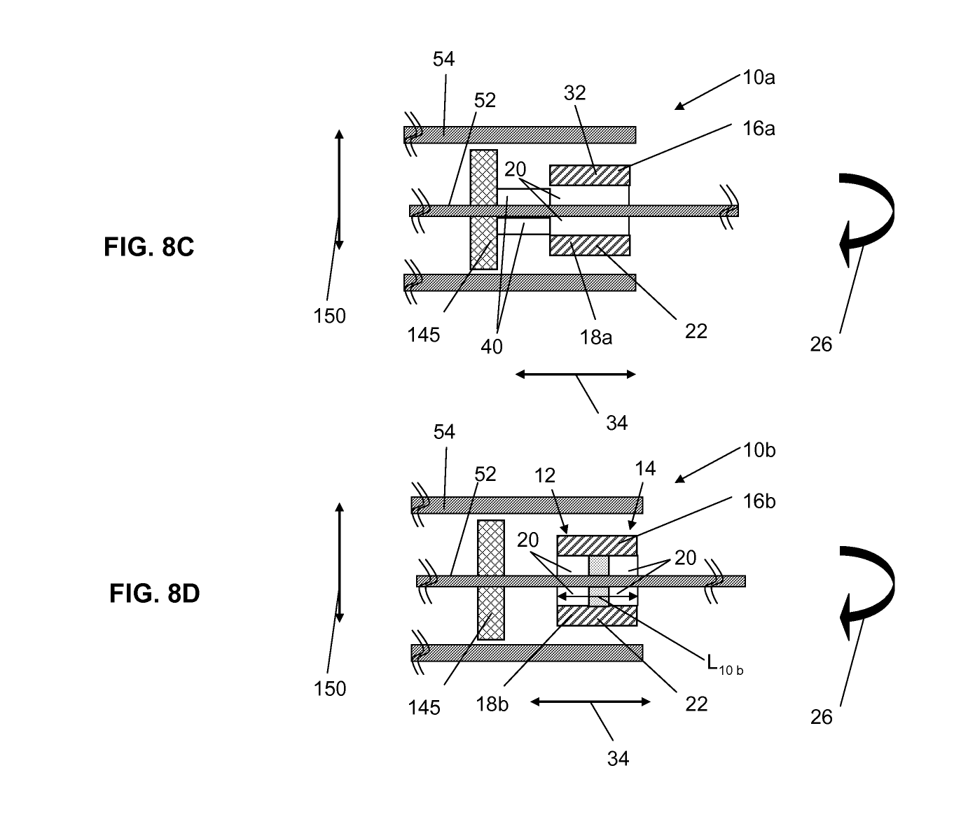

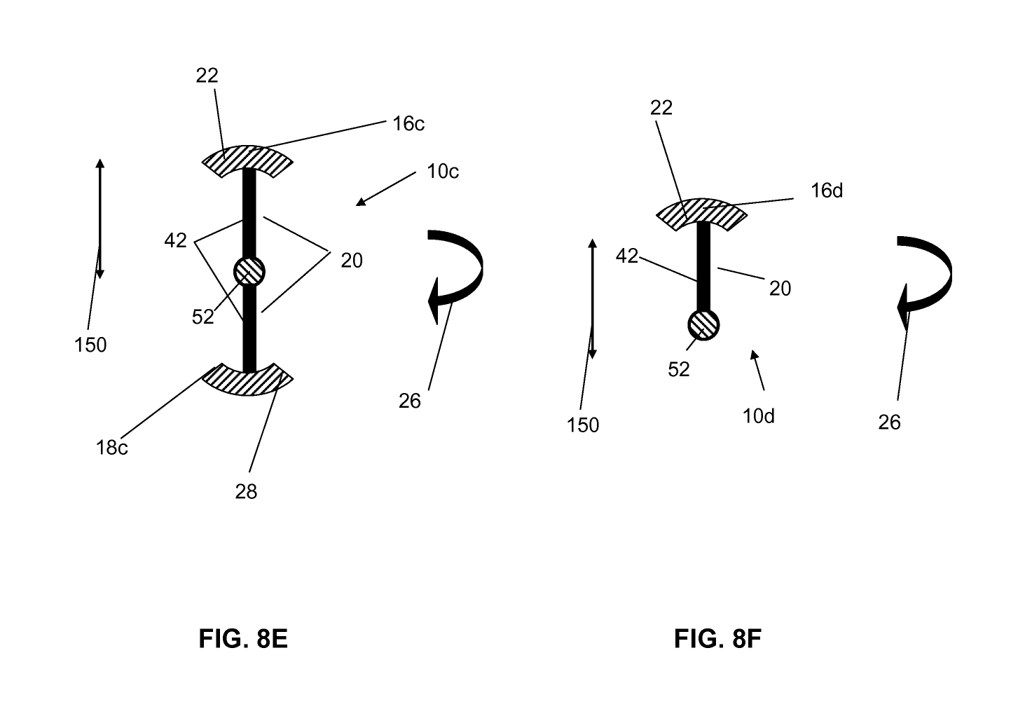

The release device 100 comprises a retaining body 10 for retaining the implant 102 in the insertion device 130. In addition, the retaining body 10 has a proximal end 12, which, in the state of use, is distant from the distal end 140 of the insertion device 130, and a distal end 14, which, in the state of use, faces the distal end 140 of the insertion device 130. The retaining body 10 is formed by a bushing 30, the cylindrical jacket 32 of which extends in the circumferential direction 26 about the inner insertion element 52 and in the axial direction 34 parallel thereto (see also FIG. 8A). Furthermore, the retaining body 10 comprises a passage 36 for the inner insertion element 32. The retaining body 10 is fastened or adhered by way of the distal end 14 thereof to the inner insertion element 52, and, in fact, in the axial direction 34 between a stopper 145 and the distal end 140 or the catheter tip 125 of the insertion device 130. This takes place by way of a UV-curable adhesive, for example. This adhesive forms a base 38 of the bushing 30. The cylindrical jacket 32 is made of a metal that is visible to x-rays, such as tantalum, gold or platinum. By way of an x-ray device, which is not shown here, it is therefore possible to monitor is progress, during implantation of the implant 102, of the inner insertion element 52 and, therefore, the implant 102 using the insertion device 130 in the direction of insertion 56, and to monitor correct positioning of the implant 102 at an implantation site.

For interaction with the implant 102, the retaining body 10 comprises two segments 16, 18, which are distributed evenly in the circumferential direction 26. The two segments 16, 18 are designed as a single piece with the cylindrical jacket 32 of the bushing 30 and, therefore, are formed on the same component 22, i.e. in a region 24 of the component 22 that extends in the circumferential direction 26 about the inner insertion element 52. In addition, each of the segments 16, 18 radially delimits a space 20, and so the space 20 extends in a radial direction 150 in each case between the inner insertion element 52 and the particular segment 16, 18. Furthermore, this space 20 is disposed at the proximal end 12 of the retaining body 10 in each case, and forms an open cavity at a proximal end 12 of the retaining body 10.

For interaction with the retaining body 10, the implant 102 comprises two retaining elements 104, which are distributed evenly around a circumference of the implant 102. Each of the retaining elements 104 comprises two regions 108 and 120 and are in the general form of hooks. The retaining element 104 is shown in detail in FIG. 1B. The region 120 is disposed or integrally formed at a proximal end 106 of the implant 102. In addition, the region 120 extends substantially in the radial direction 150 toward an inner axis 116 of the implant 102. The region 120 is therefore disposed at an angle α of approximately 90° relative to a region 114 of the implant 102 that extends substantially parallel to the inner axis 116 of the implant 102. The region 108 is disposed at a radial end of the region 120 or is connected thereto and forms an angle β therewith of approximately 90°. The region 108 therefore extends substantially parallel to the inner axis 116 of the implant 102 and the region 114. Due to the angular placement of the regions 108, 114 and 120, the retaining element 104 has a directional change 112 relative to the region 114 that forms a total angle Σ of more than 90° or approximately 180°.

The segments 16, 18 and the space 20 of the retaining body 10, and the regions 108 and 120 of the implant 102 are matched to one another in such a way that, in the interaction of the retaining element 104 with the retaining body 10, the region 108 of the retaining element 104 can be inserted into the space 20, in the direction of insertion 56, and/or can be withdrawn from the space 20, against the direction of insertion 56.

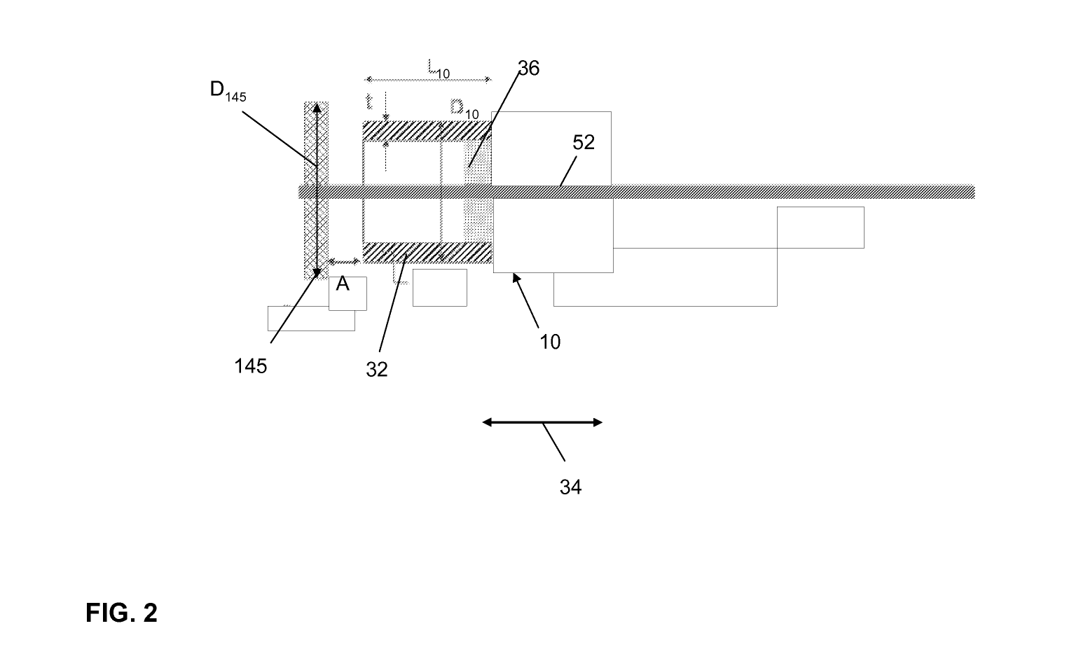

FIG. 2 shows the retaining body 10 and the stopper 145 in a detailed depiction. The dimensions mentioned in the following apply for an 18F design. An outer diameter D10 of the retaining body 10 is approximately 4 mm. Since a radial distance Ar between the outer shaft and the inner shaft is approximately 2.1 mm, there is sufficient space available for placing the retaining element 10 in the insertion device 130 (cf. FIG. 1A). A material thickness t of the retaining body 10 depends on the material and is 0.2 mm, for example. An axial length L10 of the retaining body 10 is 5 mm, for example. The stopper 145, which is made of polyamide, for example, has an outer diameter D145 of 5.3 mm, for example. An axial distance A between the stopper 145 and the retaining body 10 is 2 mm, for example. A wall thickness W of the implant 102 or the retaining element 104 is 0.5 mm, for example (see FIG. 1B). An axial length La of the retaining element 104 or the region 108 is 1 mm, for example, and a radial length Lr of the retaining element 104 or the region 120 is greater than the material thickness t of the segment 16, 18 of the retaining body 10 and, for example, is twice the material thickness t of 0.2 mm, and is therefore 0.4 mm. The dimensions indicated in FIGS. 1B and 2 are not true to scale.

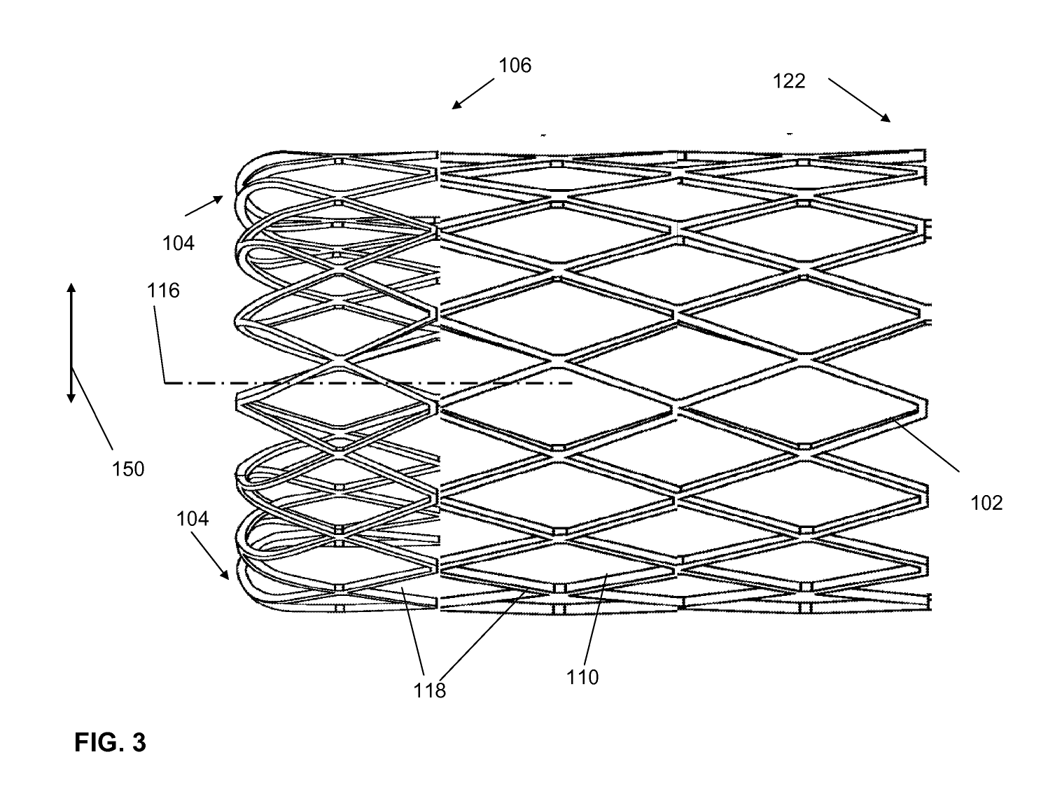

FIG. 3 shows the proximal end 106 of the implant 102 in a detailed depiction. The implant 102, which is a stent or an artificial heart valve implant, for example, is designed to be self-expanding. In addition, the implant 102 comprises a base body 110, which has a basic structure 118 in the form of a wire mesh. The retaining elements 104 comprise this basic structure 118 by least more than 50% and, in fact, up to 100%. According to a method for manufacturing the implant 102, the proximal end 106 of the implant 102 is curved in the radial direction 150 toward the inner axis 116 of the implant 102. The retaining elements 104 are formed as a result. It is also possible, in principle, for only one segment of the proximal end 106 or the proximal end 106 to be bent inwardly only partially.

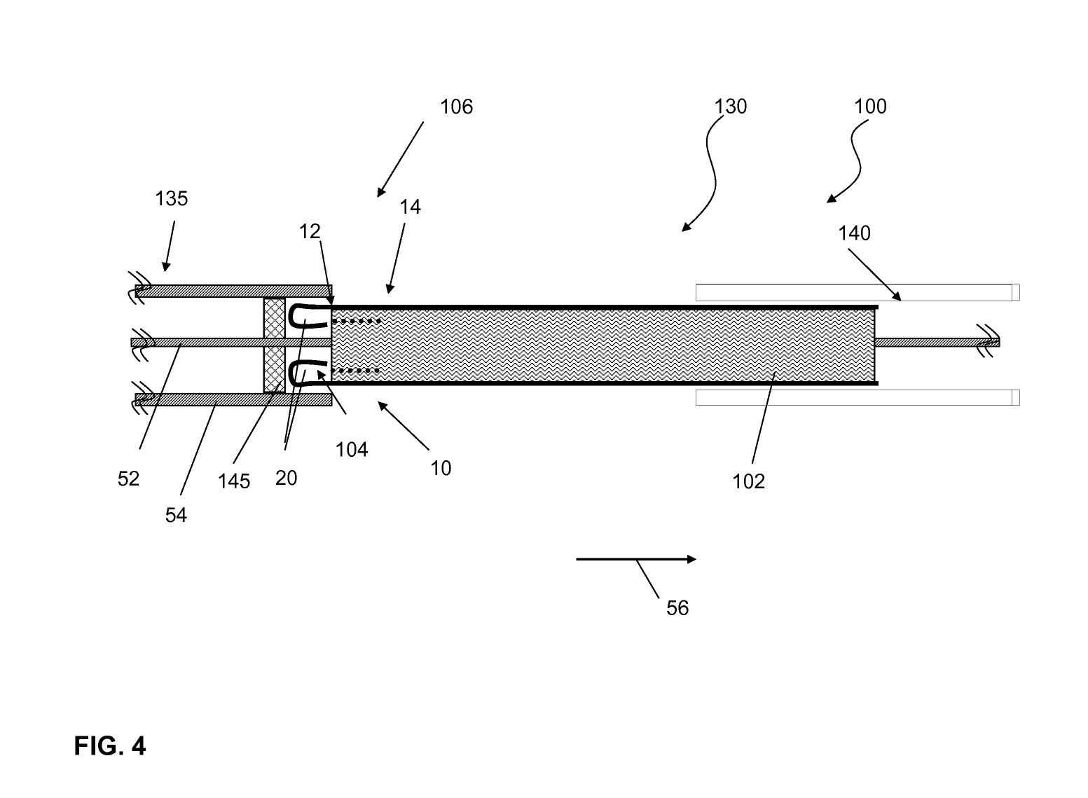

A method for connecting the implant 102 by way of the retaining body 10 and, therefore, the insertion device 130, will now be described with reference to FIG. 1A and FIGS. 4 to 6. In a first step, the previously cooled, crimped implant 102 is placed at/on the inner insertion element 52 against the direction of insertion 56. In so doing, the entire implant 102 is slid by way of the inner insertion element 52 and the proximal end 106 of the implant 102 comprising the retaining elements 104 over the retaining body 10, and therefore at least the proximal end 106 of the implant 102 is disposed proximally with respect to the retaining body 10. The sliding motion continues, for example, until the proximal end 106 of the implant 102 or the regions 120 of the retaining elements 104 impact the stopper 145 (see FIG. 4). The stopper 145 therefore limits the motion of the implant 102 in the direction of the proximal end 135 of the insertion device 130. Care must be taken in advance, of course, to ensure that the distance A between the stopper 145 and the retaining body 10 is at least wider than the particular axial length La of the retaining element 104 or the region 108.

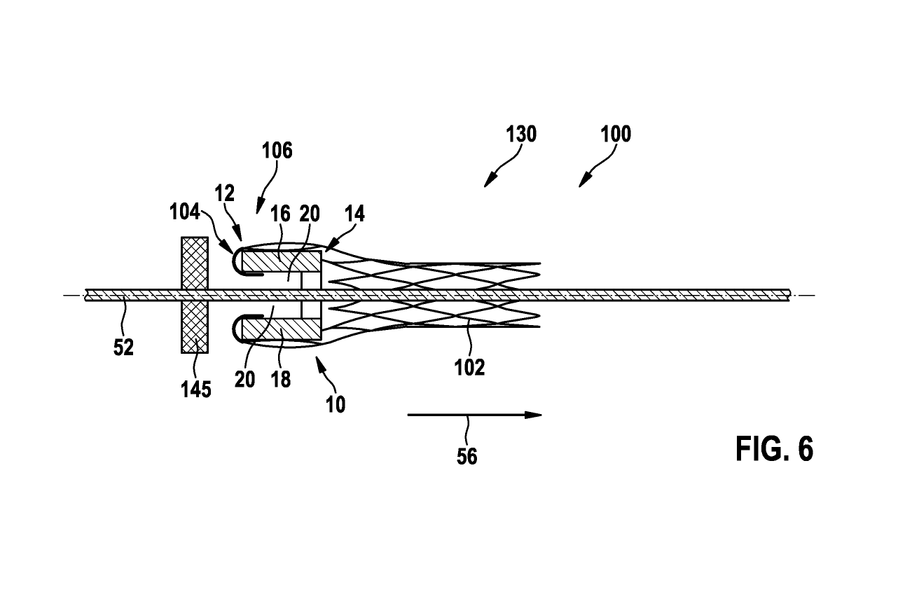

In a second step, the outer insertion element 54 is slid in the direction of insertion 56, whereby the implant 102 likewise moves in the direction of insertion 56 due to static friction between the insertion element 54 and the implant 102. As a result, the region 108 of each of the retaining elements 104 of the proximal end 106 of the implant 102 is inserted, in the direction of insertion 56, into the space 20 that is radially delimited by the retaining body 10 (see FIGS. 5 and 6). The retaining body 10 comprising the implant 102 is thereby placed in the outer insertion element 54, and assembly is complete.

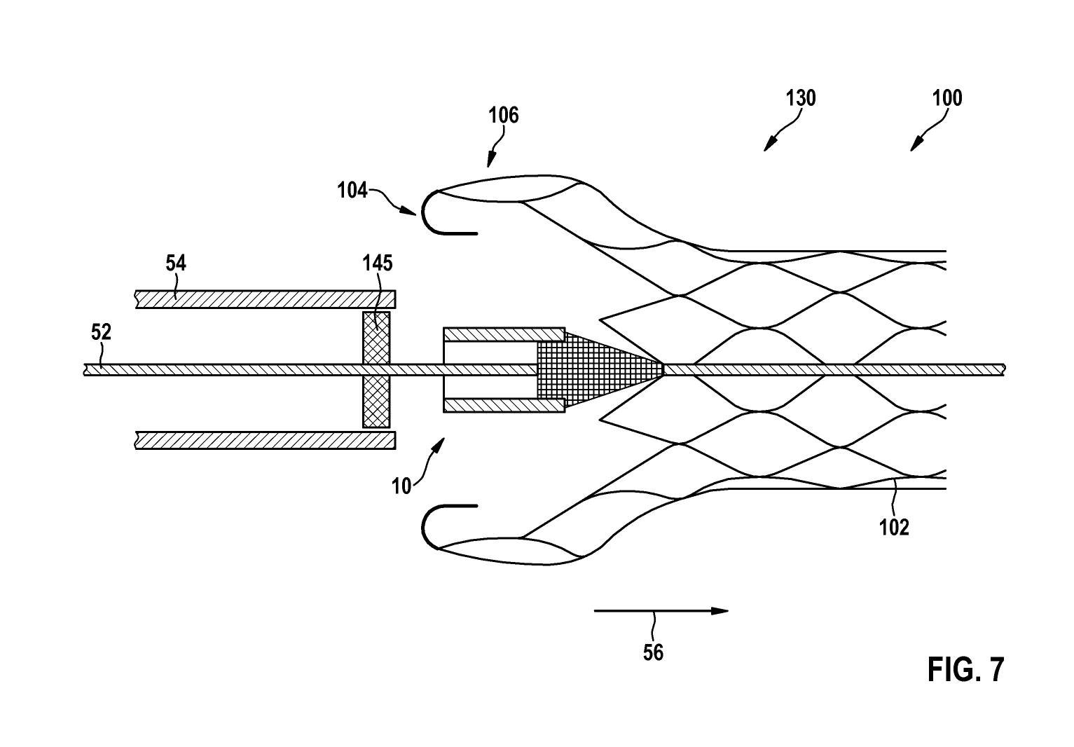

In order to implant the implant 102 in the body, the insertion device 130 prepared in this manner is introduced into the body. The motion of the outer insertion element 54 against the direction of insertion 56 induces release of the distal end 122 of the implant 102, which then opens and positions itself due to the self-expansion capability thereof. In this stage, the implant 102 is still securely connected by way of the proximal end 106 thereof to the retaining body 10. Functionality tests of the implant 102 can now be carried out, if necessary. If a malfunction is found, the implant 102 can be removed from the body. It would also be possible to reposition the implant 102 if it is incorrectly positioned. When the outer insertion element 54 moved, the implant 102 or the proximal end 106 of the implant 102 comprising the regions 108 was moved against the direction of insertion 56, thereby releasing them from the space 20 that is radially delimited by the retaining body 10. The movement continues, for example, until the proximal end 106 impacts the stopper 145. This impact against the stopper 145 induces ejection of the implant 102. As a result, the proximal end 106 of the implant 102 also opens due to the radial force thereof; this situation is shown in FIG. 7. Next, the release device 100 or the retaining body 10 comprising the inner shaft is retracted into the outer shaft, and the insertion device 130 is removed from the body. The implant 102 remains permanently positioned in the body (not shown).

As an alternative, a second type of release of the implant 102 is provided. In this case, each of the regions 108 of the retaining elements 104 remains in the space 20 after the outer insertion element 54 is moved against the direction of insertion 56. Release is achieved in this case by moving the inner insertion element 52 in the direction of insertion 56 by approximately 5 mm, whereby the regions 108 are released from the spaces 20 and the implant 102 expands automatically.

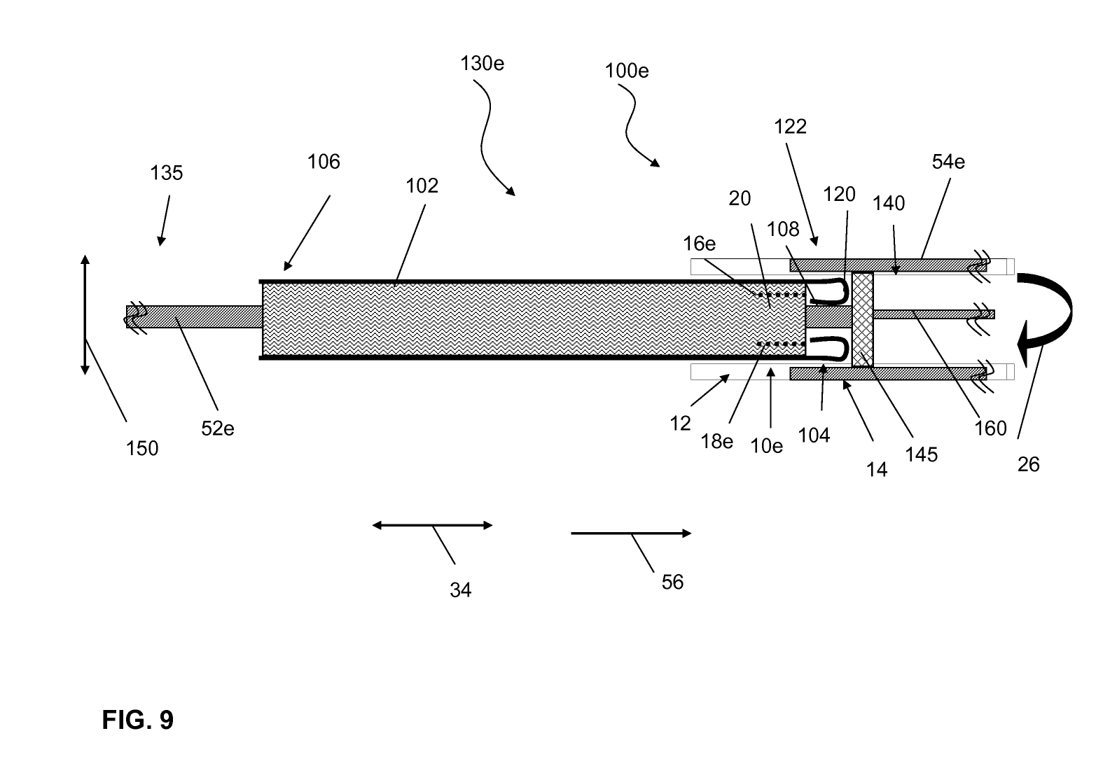

FIGS. 8B-F show four alternative exemplary embodiments of the retaining body 10, and FIGS. 9-11 show an alternative exemplary embodiment of the release device 100 comprising the retaining body 10 and the insertion device 130. Components, features, and functions that are essentially the same are labeled using the same reference characters. To differentiate the exemplary embodiment shown in FIGS. 8B-F and 9-11 from that shown in FIGS. 1A to 8A, however, the letters “a” to “e” are appended to the reference characters for the deviating components shown in the exemplary embodiment in FIGS. 8B-F and 9-11. The description that follows is limited mainly to the differences from the exemplary embodiment presented in FIGS. 1A through 8A, and reference can be made to the description of the exemplary embodiment shown in FIGS. 1A through 8B with regard for the components, features, and functions that remain the same.

The retaining body 10a shown in FIGS. 8B and 8C differs from the retaining body 10 depicted in FIGS. 1A to 8A in that the retaining body 10a is fastened or bonded to a is stopper 145 by way of webs 40, which extends in the axial direction 34 as shown in the view (cf. FIG. 8C). To ensure unobstructed interaction of segments 16a, 18a of the retaining body with retaining elements of an implant that is not shown here, the webs 40 are disposed in the circumferential direction 26 offset with respect to the segments 16a, 18a. Furthermore, the retaining body 10 is therefore designed merely as a cylindrical jacket 32, without a base, and therefore spaces 20 are cavities having openings at two sides (cf. FIG. 8B).

The retaining body 10b depicted in FIG. 8D differs from the retaining body 10 shown in FIGS. 1A to 8A in that the retaining body 10b has spaces 20 at a proximal end 12 and at a distal end 14, and the retaining body 10b is therefore fastened to an inner insertion element 52 in the center in the axial direction 34 with respect to the length L10b thereof.

The retaining body 10c depicted in FIG. 8E differs from the retaining body 10 shown in FIGS. 1A to 8A in that the retaining body 10c comprises two segments 16c, 18c that are formed on two different components 22, 28. For connecting or attaching the segments 16c, 18c to an inner insertion element 52, the retaining body 10c comprises bars 42, which extend in the radial direction 150.

The retaining body 10d shown in FIG. 8F differs from the retaining body 10 depicted in FIGS. 1A to 8A in that the retaining body 10d comprises a segment 16d, which is connected to an inner insertion element 52 by way of a bar 42, which extends in the radial direction 150.

FIG. 9 shows a longitudinal view through an alternative exemplary embodiment of a release device 100e of an insertion device 130e, which is only partially shown. The insertion device 130e is, for example, a catheter comprising a shaft region having two coaxially disposed insertion elements 52e, 54e, e.g. an inner shaft (insertion element 52e) and, enclosing it, an outer shaft (insertion element 54e), which can be enclosed by an outer sleeve, which is not shown. In the state of use by an operator, i.e. during fastening of the implant 102 to the release device 100e or during implantation, the insertion device 130e faces an operator by way of the proximal end 135 thereof. The implant 102 is placed on the is distal end 140 of the shaft region between the inner shaft and the outer shaft, and is intended for release at the implantation site in the animal body or human body. The implant 102 is placed around the inner insertion element 52e, for example, and is released in a direction of insertion 56 starting at a proximal end 106 of the implant 102 by way of a relative motion between the first insertion element 52e and the second insertion element 54e. The outer insertion element 54e is connected to the catheter tip 125, but the inner insertion element 52e is not. To connect the proximal end 135 of the insertion device 130e and the catheter tip 125, the guide device 130e comprises a guide element 160, which extends coaxially to the inner insertion element 52e and extends therein, and is formed, for example, by a shaft having an insertion wire and a lumen.

The release device 100e comprises a retaining body 10e for retaining the implant 102 in the insertion device 130e. The retaining body 10e comprises a proximal end 12, which, in the state of use, is distant from the distal end 140 of the insertion device 130e, and a distal end 14, which, in the state of use, faces the distal end 140 of the insertion device 130e. The retaining body 10e is formed by a bushing 30, the cylindrical jacket 32 of which extends in the circumferential direction 26 about the inner insertion element 52e and in the axial direction 34 parallel thereto (analogous to FIG. 8A). The retaining body 10e is fastened or adhered to the inner insertion element 52e by way of the proximal end 12 thereof and, in fact, in the axial direction 34 between a stopper 145 and the proximal end 135 of the insertion device 130e.

For interaction with the implant 102, the retaining body 10e comprises two segments 16e, 18e that are distributed evenly in the circumferential direction 26. The segments 16e, 18e radially delimit a space 20, and so the space 20 extends in a radial direction 150 in each case between the inner insertion element 52e and the particular segment 16e, 18e. Furthermore, this space 20 is disposed at the distal end 14 of the retaining body 10e in each case, and forms a cavity that is open at a distal end 14 of the retaining body 10e (not shown here in detail, analogous to FIG. 1A).

For interaction with the retaining body 10e, the implant 102 comprises two retaining elements 104, each of which forms two regions 108 and 120, which form a hook. The segments 16e, 18e and the space 20 of the retaining body 10e, and the regions 108 and 120 of the implant 102 are matched to one another in such a way that, in the interaction of the retaining element 104 with the retaining body 10e, the region 108 of the retaining element 104 can be inserted into the space 20, in the direction of insertion 56, and/or can be withdrawn from the space 20, against the direction of insertion 56. The implant 102 is analogous in design to the implant 102 of the exemplary embodiment of FIGS. 1A to 8A, and is manufactured in a analogous manner, wherein the retaining elements 104 are formed on the distal end 122 (cf. also FIG. 3).

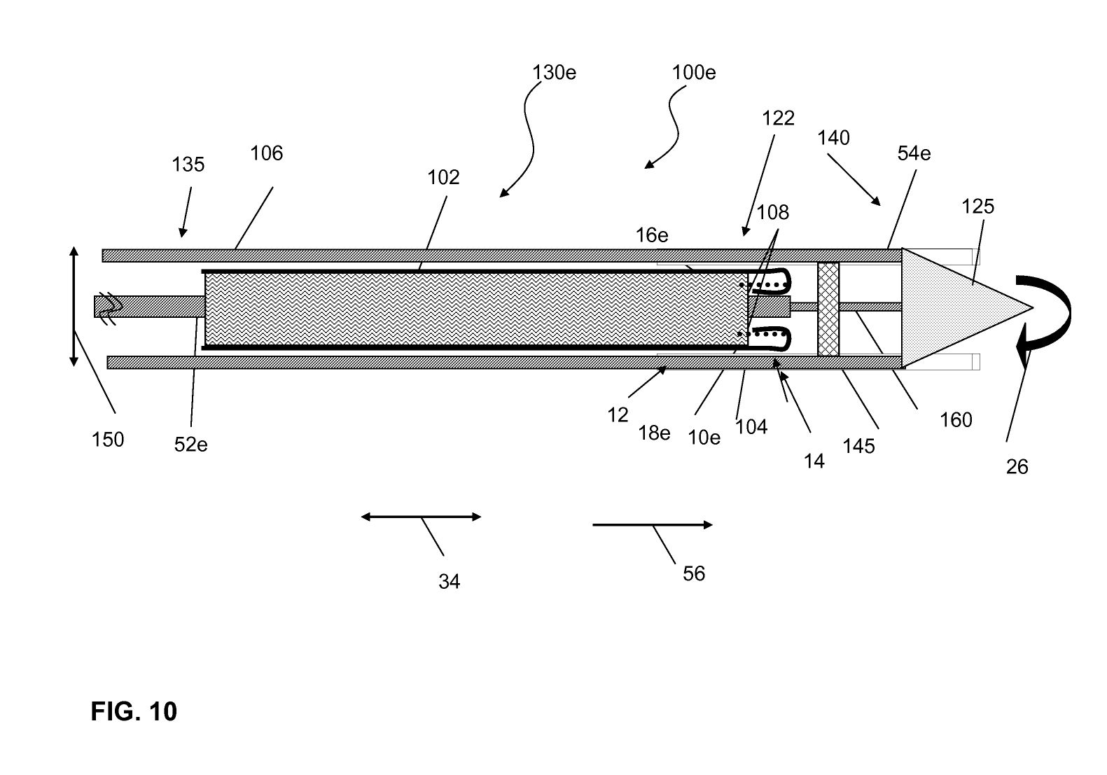

A method for connecting the implant 102 by way of the retaining body 10e and, therefore, the insertion device 130e, will now be described with reference to FIGS. 9 and 10. In a first step, the previously cooled, crimped implant 102 is placed in the direction of insertion 56 at/on the inner insertion element 52e. In so doing, the entire implant 102 is slid by way of the inner insertion element 52e and the distal end 122 of the implant 102 comprising the retaining elements 104 over the retaining body 10e, and therefore at least the distal end 122 of the implant 102 is disposed distally with respect to the retaining body 10e. The sliding motion takes place, for example, until the distal end 122 of the implant 102 or the regions 120 of the retaining elements 104 impact the stopper 145 (see FIG. 9). The stopper 145 therefore limits the motion of the implant 102 in the direction of the distal end 140 of the insertion device 130e.

As an alternative, it would also be possible to place the implant 102 on the insertion element 52e against the direction of insertion 56. In so doing, the entire implant 102 is slid over the inner insertion element 52e and the stopper 145, wherein at least the distal end 122 comprising the retaining elements 104 of the implant 102 is disposed distally with respect to the retaining body 10e.

In a second step, the outer insertion element 54e is slid against the direction of insertion 56, whereby the implant 102 likewise moves against the direction of insertion 56e due to static friction between the insertion element 54e and the implant 102. As a result, the region 108 of the retaining elements 104 of the distal end 122 of the implant 102 is inserted, in the direction of insertion 56, into the space 20 that is radially delimited by the retaining body is 10e (see FIG. 10). As a result, the retaining body 10e comprising the implant 102 is placed in the outer insertion element 54e, and assembly is complete.

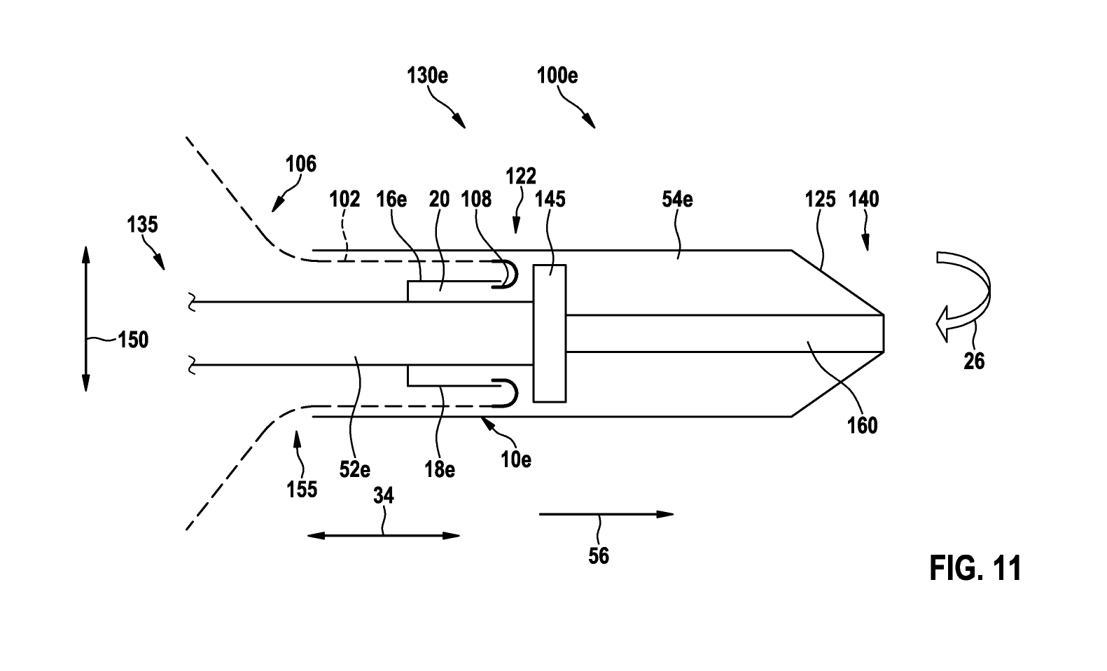

In order to implant the implant 102 in the body, the insertion device 130e prepared in this manner is introduced into the body. By moving the catheter tip 125 and the outer insertion element 54e by way of the guide element 160 in the direction of insertion 56, an opening 155 is exposed on the outer insertion element 54e, which extends in the circumferential direction 26, thereby releasing a proximal end 106 of the implant 102, which can emerge from the outer insertion element 54e through the opening 155. As a result, the proximal end 106 of the implant 102 opens and positions itself (cf. FIG. 11). In this stage, the implant 102 is still securely connected by way of the distal end 122 thereof to the retaining body 10e. It is now possible to perform functionality tests of the implant 102, or to remove or reposition the implant 102. The connection between the proximal end 135 of the insertion device 130e and the catheter tip 125 is ensured by way of the guide element 160. When the outer insertion element 54e moved, the implant 102 or the distal end 122 of the implant 102 comprising the regions 108 was moved in the direction of insertion 56, thereby releasing them from the space 20 that is radially delimited by the retaining body 10e. The movement continues, for example, until the distal end 122 impacts the stopper 145. This impact against the stopper 145 induces ejection of the implant 102. As a result, the distal end 122 of the implant 102 also opens due to the radial force thereof. Next, the catheter tip 125 and the part of the outer insertion element 54e connected thereto is retracted once more by way of the guide element 160 over the release device 100e or the retaining body 10e, whereby the opening 155 closes once more. The insertion device 130e can now be removed from the body. The implant 102 remains permanently positioned in the body (not shown).

As an alternative, a second type of release of the implant 102 would also be possible. In this case, each of the regions 108 of the retaining elements 104 remains in the space 20 after the outer insertion element 54e is moved in the direction of insertion 56. Release is achieved in this case by moving the inner insertion element 52e against the direction of insertion 56 by approximately 5 mm, whereby the regions 108 are released from the spaces 20 and the implant 102 expands automatically.

It will be apparent to those skilled in the art that numerous modifications and variations of the described examples and embodiments are possible in light of the above teaching. The disclosed examples and embodiments are presented for purposes of illustration only. Other alternate embodiments may include some or all of the features disclosed herein. Therefore, it is the intent to cover all such modifications and alternate embodiments as may come within the true scope of this invention.

1. A release device for releasing a medical implant from an insertion device, in which the implant can be released first at a distal end of the implant by way of a relative motion between a first insertion element and a second insertion element in a direction of insertion, comprising:
a retaining body for retaining the implant in the insertion device, having a proximal end, which, in the state of use, is distant from a distal end of the insertion device, and a distal end, which, in the state of use, faces the distal end of the insertion device,
wherein the retaining body has at least one segment that radially delimits at least one space that extends between one of the insertion elements and the at least one segment,
wherein the implant has at least one retaining element at a proximal end thereof, the retaining body configured to interact with the at least one retaining element, and
wherein at least one region of the at least one retaining element, upon interaction with the retaining body, is inserted into the at least one space in a direction of insertion and is withdrawn from the at least one space against the direction of insertion.

2. A release device for releasing a medical implant from an insertion device, in which the implant can be released first at a proximal end of the implant by way of a relative motion between a first insertion element and a second insertion element in a direction of insertion, comprising:
a retaining body for retaining the implant in the insertion device, having a proximal end, which, in the state of use, is distant from a distal end of the insertion device, and a distal end, which, in the state of use, faces the distal end of the insertion device,
wherein the retaining body has at least one segment that radially delimits at least one space that extends between one of the insertion elements and the at least one segment,
wherein the implant has at least one retaining element at a distal end of the retaining element the retaining body configured to interact with the at least one retaining element, and
wherein at least one region of the at least one retaining element, upon interaction with the retaining body, is inserted into the at least one space in a direction of insertion and is withdrawn from the at least one space against the direction of insertion.

3. The release device according to claim 1, wherein the at least one space is disposed at one of the proximal end of the retaining body or at the distal end of the retaining body.

4. The release device according to claim 1, wherein the retaining body is fastened to at least one of a distal end and a proximal end of at least one of the insertion elements.

5. The release device according to claim 1, wherein the retaining body is adhered to an inner insertion element by a UV-curable adhesive.

6. The release device according to claim 1, wherein the retaining body comprises at least two segments, which radially delimit at least one space that extends between an inner insertion element and each of the at least two segments.

7. The release device according to claim 6, wherein the at least two segments are formed on a same component and are disposed in a region of a component that extends in a circumferential direction about one of the insertion elements.

8. The release device according to claim 6, wherein the at least two segments are formed on two different components.

9. The release device according to claim 1, wherein the retaining body comprises a material that is selected from the group consisting of: a ceramic, a glass, a rubber, a plastic, a metal, a metal that is visible to x-rays, polyamide, acrylnitrile butadiene styrene, steel, Nitinol, tantalum, gold or platinum.

10. The release device according to claim 1 and further including the insertion device for inserting a medical implant.

11. The release device according to claim 2 and further including the insertion device for inserting a medical implant.

12. The release device according to claim 10, wherein the insertion device further comprises a stopper which is operative to limit motion of the implant in one of a direction of a proximal end of the insertion device or in a direction of the distal end of the insertion device, and induces ejection of the implant.

13. The release device according to claim 10, and further including the medical implant, and where the medical implant comprises:
a base body;
at least one retaining element configured for interaction with the retaining body of the release device, wherein the at least one retaining element has at least one change in direction, which forms a total angle of more than 90° relative to a region of the base body that extends substantially parallel to an inner axis of the base body, and is oriented in the radial direction with respect to the inner axis, wherein at least one region of the at least one retaining element, upon interaction with the retaining body, is inserted into the at least one space of the retaining body in the direction of insertion and is be withdrawn from the at least one space against the direction of insertion.

14. The release device according to claim 11, and further including the medical implant, and where the medical implant comprises:
a base body;
at least one retaining element configured for interaction with the retaining body of the release device, wherein the at least one retaining element has at least one change in direction, which forms a total angle of more than 90° relative to a region of the base body that extends substantially parallel to an inner axis of the base body, and is oriented in a radial direction with respect to the inner axis, and wherein at least one region of the at least one retaining element, upon interaction with the retaining body, is inserted into at least one space of the retaining body against the direction of insertion and/ is withdrawn from the at least one space in the direction of insertion.

15. The release device according to claim 13, wherein the medical implant base body has at least one basic structure, and wherein the at least one retaining element comprises the basic structure of the base body at least by more than 50%.

16. The release device according to claim 13, wherein the at least one retaining element of the medical implant has a region that extends substantially in a radial direction toward an inner axis of the implant and has a length that is greater than a thickness of a segment of the retaining body of the release device.

17. The release device according to claim 13, wherein the medical implant is a self-expanding implant.

18. The release device according to claim 13, wherein the release device is configured for connecting to the medical implant using the retaining body of the release device in the insertion device, through the following steps:
placing at least the proximal end of the implant, against the direction of insertion, at one of the insertion elements, so that at least the proximal end of the implant is disposed proximally with respect to the retaining body;
moving at least the proximal end of the implant in the direction of insertion, so that at least one region of at least one retaining element of the proximal end of the implant is inserted in the direction of insertion into a space that is radially delimited by the retaining body; and
placing the retaining body comprising the implant in at least one of the insertion elements.

19. A method for connecting the release device according to claim 14 to the medical implant of claim 14, using the retaining body of the release device in the insertion device, the method comprising the following steps:
placing at least the distal end of the implant, in the direction of insertion, at one of the insertion elements, so that at least the distal end of the implant is disposed distally with respect to the retaining body;
moving at least the distal end of the implant against the direction of insertion, so that at least one region of at least one retaining element of the distal end of the implant is inserted against the direction of insertion into a space that is radially delimited by the retaining body; and
placing the retaining body comprising the implant in at least one of the insertion elements.
